(19) United States
(12) Patent Application Publication
(10) Pub. No.: US 2025/0243137 A1
(43) Pub. Date: Jul. 31, 2025

(54) METHOD FOR PREPARING 3-CHLOROBICYCLO[3.2.1]-3-OCTEN-2-OL

(71) Applicant: SHANDONG WEIFANG RAINBOW CHEMICAL CO.,LTD, Shandong (CN)

(72) Inventors: Hongquan ZHANG, Shandong (CN); Zhiqing LI, Shandong (CN); Zongtao HAN, Shandong (CN); Yongquan YU, Shandong (CN); Guangli ZHAO, Shandong (CN)

(73) Assignee: SHANDONG WEIFANG RAINBOW CHEMICAL CO.,LTD, Shandong (CN)

(21) Appl. No.: 19/182,515

(22) Filed: Apr. 17, 2025

Related U.S. Application Data
(63) Continuation of application No. PCT/CN2023/126278, filed on Oct. 24, 2023.

(30) Foreign Application Priority Data
Jan. 19, 2023 (CN) 202310074577.6

(51) Int. Cl. C07C 29/124; C07C 35/52

(52) U.S. Cl. CPC C07C 29/124; C07C 35/52; C07C 2602/44

(57) ABSTRACT

Disclosed is a method for preparing 3-chlorobicyclo[3.2.1]-3-octen-2-ol, which belongs to the field of pharmaceutical technology. The method for preparing 3-chlorobicyclo[3.2.1]-3-octen-2-ol can be achieved by any of the following reaction routes: Reaction route I: 3,4-dichlorobicyclo[3.2.1]-2-octene is esterified in the presence of a carboxylate, and hydrolyzed in a strong base to obtain 3-chlorobicyclo[3.2.1]-3-octen-2-ol; Reaction route II: in the presence of an inorganic salt of strong base and weak acid, 3,4-dichlorobicyclo[3.2.1]-2-octene is reacted in a solvent to obtain 3-chlorobicyclo[3.2.1]-3-octen-2-ol. The present application can result in decreased formation of polymers and impurities, more obvious layering, and improved purification efficiency.

### CROSS-REFERENCE TO RELATED APPLICATION

This application is a continuation of international application of PCT application serial no. PCT/CN2023/126278, filed on Oct. 24, 2023, which claims the priority benefit of China application no. 202310074577.6, filed on Jan. 19, 2023. The entirety of each of the above-mentioned patent applications is hereby incorporated by reference herein and made a part of this specification.

### BACKGROUND

### Technical Field

The present application relates to the field of pharmaceutical technology, and in particular to a method for preparing 3-chlorobicyclo[3.2.1]-3-octen-2-ol.

### Description of Related Art

3-chlorobicyclo[3.2.1]-3-octen-2-ol (formula I) is used as an intermediate in the synthesis of common pesticide herbicides. The compounds synthesized therefrom have certain herbicidal activity, such as bicyclopyrone: 4-hydroxy-3-{2-[(2-methoxyethoxy)methyl]-6-(trifluoromethyl)-3-pyridinylcarbonyl}bicyclo[3.2.1]oct-3-en-2-one, and benzobicylon: 3-(2-chloro-4-methylsulfonylbenzoyl)-2-phenylthiobicyclo[3,2,1]oct-2-en-4-one. Both bicyclopyrone and benzobicylon are herbicides of the 4-hydroxyphenylpyruvate dioxygenase (HPPD) inhibitor.

Bicyclopyrone, developed by Syngenta, has good activity against broadleaf weeds as well as perennial and annual weeds. It can be used in corn, wheat, barley, sugarcane and other crop fields pre- or post-seedling. It provides excellent control of resistant weeds and other recalcitrant broadleaf weeds, especially those that have been resistant to ALS-inhibiting herbicides, growth-hormone herbicides and glyphosate.

Benzobicylon, developed by SDS Biotech K.K., can be absorbed by the roots and stems of weeds and then transported throughout the plant, and it primarily inhibits the biochemical synthesis of carotenoids in photosynthetic pigments, resulting in a decreased content, leaf whitening and eventual weed death. Benzobicylon is highly selective between rice and weeds, safe for rice, highly compatible with the environment, and has low toxicity to fish and mammals. Furthermore, it exhibits a broad herbicidal activity and a long persistence period.

Current methods for synthesizing this compound of formula I are:

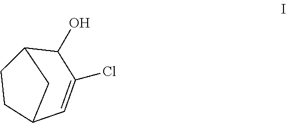

1) Kaenel, Hans Ruedi; Ganter, Camille, The novel adamantine isomer tricyclo[4.4.0.0^{3,9}]decane(2-homotwistbrendane) [J] Helvetica Chimica Acta (1985), 68(5), 1226-34, wherein, a similar structure is provided, and a potassium hydroxide solution is used for hydrolysis.

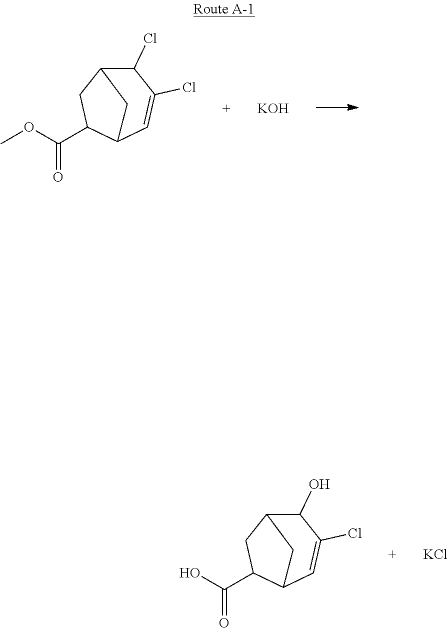

In Route A-1, hydrolysis is performed with a potassium hydroxide solution, yielding only 82%.

2) 3-chlorobicyclo[3.2.1]-3-octen-2-ol is obtained by hydrolysis of 3,4-dichlorobicyclo[3.2.1]-2-octene as a raw material, which is described in the following three articles: CN1440376A, CN105693569A, and Ge, Fa-xiang, Synthesis of post-emergence paddy herbicide benzob[J]Anhui Huagong (2013), 39(6), 41-43, as shown in Route A-2.

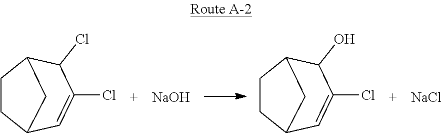

The raw material 3,4-dichlorobicyclo[3.2.1]-2-octene undergoes hydrolysis at high temperature in Route A-2, achieving a yield of approximately 94%, which, however, has a disadvantage of large consumption of alkali. In the experimental parts of the patent examples and the literature, 4 equivalents of sodium hydroxide solution are used for the reaction, resulting in difficulties in wastewater treatment. However, under these conditions, floc impurities that are insoluble in either aqueous or oil phase, are prone to be formed in Route A-2, which results in difficulties in layering during extraction and increased purification challenges. Additionally, 4,4′-oxo-bis(3-chlorobicyclo[3.2.1]-2-octene (simply, an ether impurity) is also prone to be formed as a by-product, leading to a decreased yield, which may be present in the organic phase of extraction, resulting in a low material purity generally not exceeding 90%. However, in the practical production, the purity needs to reach 94% or more to be suitable for subsequent production. Since the boiling point of the ether impurity is higher than that of the target product, rectification and other complex processes are required for further purification, which not only increases three wastes, but also raises the cost of production.

### SUMMARY

### Invention Purpose

In order to overcome the above deficiencies, the purpose of the present application is to provide a method for preparing 3-chlorobicyclo[3.2.1]-3-octen-2-ol. The present application may result in decreased formation of polymers and impurities, more obvious layering by extraction, and improved purification efficiency.

### Solutions

In order to achieve the purpose of the present application, the technical solutions of the present application are provided as follows:

In a first aspect, the present application provides a substituted bicyclol ester compound having a structural formula as shown in formula IV:

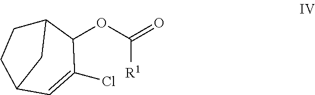

wherein, R¹ is hydrogen, C₁-C₈ alkyl, phenyl, C₁-C₈ halogen-substituted alkyl or halogen-substituted phenyl.

Further, R¹ is hydrogen, C₁-C₅ alkyl, phenyl, C₁-C₅ chloro-substituted alkyl (which may be monochloro-substituted alkyl, dichloro-substituted alkyl or trichloro-substituted alkyl) or chloro-substituted phenyl (which may be monochloro-substituted phenyl, dichloro-substituted phenyl or trichloro-substituted phenyl). Preferably, R¹ is hydrogen, methyl, ethyl, 4-chlorophenyl or phenyl.

In a second aspect, there is provided a method for preparing the substituted bicyclol ester compound, the method comprising the steps of: in the presence of a carboxylate, esterifying 3,4-dichlorobicyclo[3.2.1]-2-octene in a solvent to obtain the compound of formula IV.

In a third aspect, there is provided a method for preparing 3-chlorobicyclo[3.2.1]-3-octen-2-ol, which is obtained by using any of the following reaction routes:
Reaction route I: 3,4-dichlorobicyclo[3.2.1]-2-octene is esterified in the presence of a carboxylate and then hydrolyzed in a strong base to obtain 3-chlorobicyclo[3.2.1]-3-octen-2-ol;
Reaction route II: in the presence of an inorganic salt of strong base and weak acid, 3,4-dichlorobicyclo[3.2.1]-2-octene is reacted in a solvent to obtain 3-chlorobicyclo[3.2.1]-3-octen-2-ol.

In the preparation method of the second or third aspect, in the esterification reaction of Reaction route I, the carboxylate is selected from an alkali metal carboxylate or an alkali earth metal carboxylate, and the carboxylate is one or more selected from the group consisting of an alkali metal formate, an alkali metal acetate, an alkali metal propionate, an alkali metal 4-chlorobenzoate, an alkali metal benzoate, an alkali earth metal formate, an alkali earth metal acetate, an alkali earth metal propionate, an alkali earth metal 4-chlorobenzoate, and an alkali earth metal benzoate; or the carboxylate is one or more selected from the group consisting of sodium formate, sodium acetate, potassium acetate, calcium acetate, magnesium acetate, sodium propionate, potassium propionate, sodium 4-chlorobenzoate, potassium 4-chlorobenzoate, sodium benzoate, and potassium benzoate.

In the preparation method of the second or third aspect, the molar ratio of 3,4-dichlorobicyclio[3.2.1]-2-octene to the carboxylate in the esterification reaction of Reaction route I is 1:1-4, or 1:1.2-4, or 1:1.5-3.

In the preparation method of the second or third aspect, the reaction temperature in the esterification reaction of Reaction route I is 0° C. to a reflux temperature of each solvent, or 50° C. to a reflux temperature of each solvent, or 70° C. to a reflux temperature of each solvent, or 80° C. to a reflux temperature of each solvent, or 90° C. to a reflux temperature of each solvent, or a reflux temperature of each solvent.

In the preparation method of the second or third aspect, a catalyst is further added in a mass ratio of 0-10% of the reaction system in each reaction (including the preparation method of the second aspect, and different reactions of the two routes of the third aspect, in which the catalyst is added or not as appropriate), and the catalysts in each reaction are one or more independently or not independently selected from the group consisting of polyethylene glycol, tetrabutylammonium bromide, tetrabutylammonium chloride, benzyl trimethyl ammonium chloride, diisopropylethylamine, triethylamine, triethylenediamine, and crown ether. The types of the catalysts may be the same or different for each reaction.

In the preparation method of the second or third aspect, the esterification reaction of Reaction route I is carried out in a solvent, and the mass ratio of 3,4-dichlorobicyclo[3.2.1]-2-octene to the solvent is 1:1-20, or 1:1-12.

In the preparation method of the second or third aspect, the solvent in the esterification reaction of Reaction route I is water or an organic solvent that is one or more selected from the group consisting of dimethylformamide, dimethylacetamide, ethylene glycol monomethyl ether, ethylene glycol dimethyl ether, ethylene glycol diethyl ether, N-methyl pyrrolidone, dimethyl sulfoxide, and sulfolane. Preferably, the solvent is one or more selected from the group consisting of dimethylformamide, and dimethylacetamide. When the esterification reaction is carried out in an organic solvent, all or part or none of the organic solvent is distilled out after the esterification reaction, and the hydrolysis reaction is carried out with adding water and a strong base after the esterification reaction.

Further, in Reaction route I, a strong base is added for hydrolysis after complete esterification, alternatively, a carboxylate and a strong base are added to the reaction system at the same time for carrying out the esterification and hydrolysis reactions. Preferably, the addition of a strong base after complete esterification can result in a product with higher purity. When the carboxylate and the strong base are added at the same time, the molar ratio of the compound of formula II to the strong base (e.g., sodium hydroxide) is less than or equal to 1:1.

In the preparation method of the third aspect, the strong base in the hydrolysis reaction of Reaction route I is an alkali metal hydroxide and/or an alkali earth metal hydroxide; or, the strong base in the hydrolysis reaction of Reaction route I is one or more of sodium hydroxide, potassium hydroxide, lithium hydroxide, calcium hydroxide, and magnesium hydroxide.

In the preparation method of the third aspect, the molar ratio of 3,4-dichlorobicyclo[3.2.1]-2-octene to the strong base in the hydrolysis reaction of Reaction route I is 1:0.5-2, or 1:1-1.5.

In the preparation method of the third aspect, the inorganic salt of strong base and weak acid in Reaction route II is one or more selected from the group consisting of carbonate, phosphate, and hydrogen phosphate; alternatively, the inorganic salt of strong base and weak acid is one or more of alkali metal carbonate, alkali metal phosphate, and alkali metal hydrogen phosphate; or alternatively, the inorganic salt of strong base and weak acid is one or more of sodium carbonate, potassium carbonate, tripotassium phosphate, and dipotassium hydrogen phosphate. Sodium carbonate and/or potassium carbonate are preferable.

In the preparation method of the third aspect, the molar ratio of 3,4-dichlorobicyclo[3.2.1]-2-octene to the inorganic salt of strong base and weak acid in the hydrolysis reaction of Reaction route II is 1:1-4, or 1:1-3, or 1:1.5-3.

In the preparation method of the third aspect, water is used as a solvent in Reaction route II, and the mass ratio of 3,4-dichlorobicyclo[3.2.1]-2-octene to water is 1:1-20, or 1:1-15, or 1:3-15.

In the preparation method of the third aspect, the reaction temperature in Reaction route II is 0° C. to the reflux temperature of the solvent, or 50° C. to the reflux temperature of the solvent, or 70° C. to the reflux temperature of the solvent, or 90° C. to the reflux temperature of the solvent, or the reflux temperature of the solvent.

Reaction route I described above includes two reaction routes: Reaction route B-2, in which the esterification reaction is carried out in water; and Reaction route B-3, in which the esterification reaction is carried out under an anhydrous condition (an organic solvent). Reaction route II includes a reaction route called Reaction route B-4.

The three reaction routes B-2, B-3 and B-4 are characterized in that:

Reaction route B-2: the esterification reaction is carried out in water.

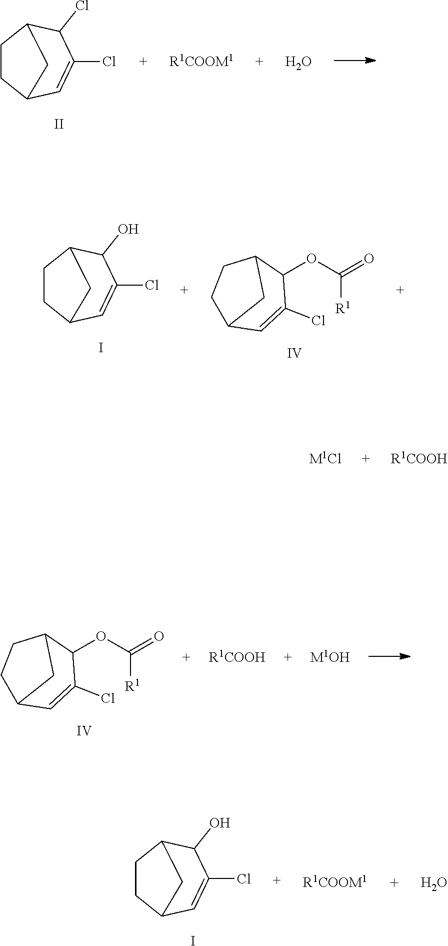

In Reaction route B-2, R¹ is hydrogen, C₁-C₈ alkyl, phenyl, C₁-C₈ halogen-substituted alkyl (preferably chloro-substituted alkyl, which may be monochloro-substituted alkyl, dichloro-substituted alkyl, or trichloro-substituted alkyl) or halogen-substituted phenyl (preferably, chloro-substituted phenyl, which may be monochloro-substituted phenyl, dichloro-substituted phenyl or trichloro-substituted phenyl); optionally, R¹ is hydrogen, C₁-C₅ alkyl, phenyl, C₁-C₅ chloro-substituted alkyl (which may be monochloro-substituted alkyl, dichloro-substituted alkyl, or trichloro-substituted alkyl), or chloro-substituted phenyl (which may be monochloro-substituted phenyl, dichloro-substituted phenyl, or trichloro-substituted phenyl); optionally, R¹ is hydrogen, methyl, ethyl, 4-chlorophenyl, or phenyl.

In Reaction route B-2, M¹ is a metal, optionally an alkali metal or alkali earth metal, preferably one or more of potassium, sodium, calcium and/or magnesium.

In Reaction route B-2, the reaction is carried out in water in the presence of a salt of strong base and weak acid (a carboxylate, preferably an alkali metal or alkali earth metal carboxylate) to give the compounds of formula I and formula IV, and then the compound of formula IV is hydrolyzed to the compound of formula I by adding an alkali metal or alkali earth metal hydroxide at a reduced temperature, which effectively reduce the formation of polymers and ether impurities (the compound of formula III). The reaction can also be carried out by adding a mixture of a carboxylate and an alkali metal or alkali earth metal hydroxide, but with a decreased yield (see Examples 2 to 13).

Reaction route B-3: the first step is carried out under an anhydrous condition in an organic solvent, followed by hydrolysis in the second step.

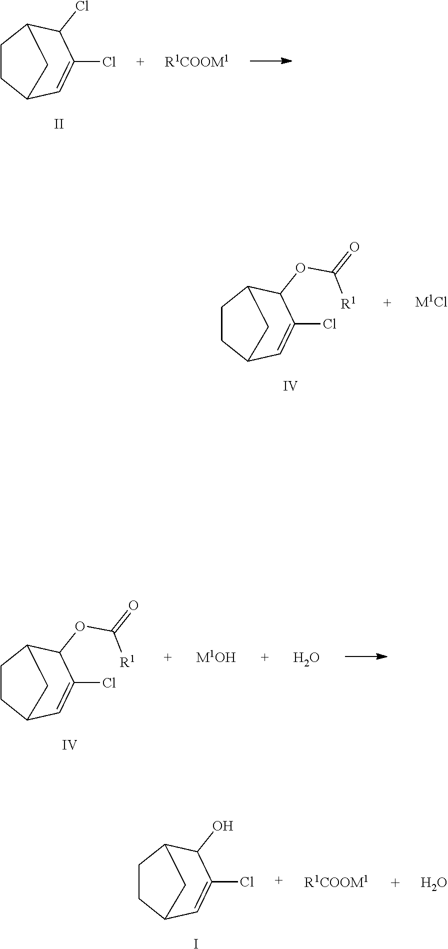

In Reaction route B-3, R¹ is hydrogen, C₁-C₈ alkyl, phenyl, C₁-C₈ halogen-substituted alkyl (preferably chloro-substituted alkyl, which may be monochloro-substituted alkyl, dichloro-substituted alkyl or trichloro-substituted alkyl) or substituted phenyl (preferably chloro-substituted phenyl, which may be monochloro-substituted phenyl, dichloro-substituted phenyl or trichloro-substituted phenyl), optionally, R¹ is hydrogen, methyl, ethyl, 4-chlorophenyl, or phenyl.

In Reaction route B-3, M¹ is a metal, optionally an alkali metal or alkali earth metal, preferably one or more of potassium, sodium, calcium and magnesium.

In Reaction route B-3, the first step of esterification is carried out under an anhydrous condition, without formation of ether impurities—the compound of formula III, after filtration of the salt and distillation of the solvent, an aqueous solution of alkali metal or alkali earth metal hydroxide is added for hydrolysis at room temperature. In this way, the hydrolysis reaction can be carried out in the aqueous solution of alkali metal hydroxide or alkali earth metal hydroxide without subjecting to a high temperature, resulting in basically free of impurity of formula III, and improved selectivity and yield (see Examples 14 to 22).

Reaction route B-4: an inorganic salt of strong base and weak acid is added for hydrolysis to directly give a compound of formula I without esterification, meanwhile reducing the formation of impurity of formula III.

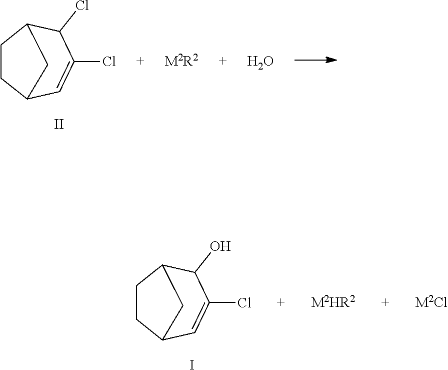

In Reaction route B-4, M² is a metal, optionally an alkali metal, preferably potassium and/or magnesium;

R² is an inorganic weak acid radical, optionally a carbonate radical, a phosphate radical, hydrogen phosphate radical.

By employing Reaction route B-4, the formation of impurity of formula III can be reduced, the reaction rate is lowered, and basically no flocs are formed in layering during extraction. However, trace amounts of impurity of formula III are still formed in a slightly lower yield compared to Reaction route B-3 (see Examples 23-32), and the distilled product can also be used directly for subsequent reactions.

### Beneficial Effects

According to the present application, 3-chlorobicyclo[3.2.1]-3-octen-2-ol can be synthesized by one way selected from Reaction routes B-2, B-3 and B-4, depending on the raw materials or solvent. Reaction routes B-2 and B-4 can achieve the following advantages by replacing the alkali type and lowering the alkali equivalent: reducing alkali waste and wastewater treatment difficulty; lowering the alkalinity of the reaction solution, thereby reducing the formation of polymers and impurities, making layering clear, and improving purification efficiency; and enabling the purity of product after distillation up to 94%, which can be used directly for subsequent reactions. The esterification reaction of Reaction route B-3 under an anhydrous condition is effective in avoiding impurity formation, and a low-temperature hydrolysis helps to prevent polymer formation. The method can be carried out under simple and mild reaction conditions with high yield, which makes it suitable for industrial production; additionally, the purity of product after distillation can reach 94%, allowing for direct use in subsequent reactions.

### BRIEF DESCRIPTION OF THE DRAWINGS

One or more examples are exemplified by the figures in the accompanying drawings that correspond thereto and the exemplifications are not intended to limit to the examples. As used herein, the word “exemplification” means “serving as an example, embodiment, or illustrative”. Any example described herein as “exemplification” is not necessarily to be construed as superior to or better than other examples.

FIG. 1 shows a mass spectrum of an analytical sample 4,4′-oxobis(3-chlorobicyclo[3.2.1]oct-2-ene) of Example 1 of the present application.

FIG. 2 shows a hydrogen spectrum of an analytical sample (3-chlorobicyclo[3.2.1]-3-octen-2-ol) of Example 1 of the present application.

FIG. 3 shows a carbon spectrum of an analytical sample (3-chlorobicyclo[3.2.1]-3-octen-2-ol) of Example 1 of the present application.

FIG. 4 shows a mass spectrum of an analytical sample (3-chlorobicyclo[3.2.1]-3-octen-2-ol) of Example 1 of the present application.

FIG. 5 shows a hydrogen spectrum of an intermediate (3-chlorobicyclo[3.2.1]-3-octen-2-acetate) of Example 1 of the present application.

FIG. 6 shows a carbon spectrum of an intermediate (3-chlorobicyclo[3.2.1]-3-octen-2-acetate) of Example 1 of the present application.

FIG. 7 shows a mass spectrum of an intermediate (3-chlorobicyclo[3.2.1]-3-octen-2-acetate) of Example 1 of the present application.

FIG. 8 shows diagrams of ethylene dichloride-extracted reaction solutions from Examples 2, 23, and Comparative Example 1 of the present application; wherein, a shows the extraction diagram of Comparative Example 1, with the aqueous and organic phases being orange-brown in color; b shows the extraction diagram of Example 2, with the aqueous phase being clear and colorless and the oil phase being yellow; and c shows the extraction diagram of Example 23, with the aqueous phase being clear and colorless, and the oil phase being yellow.

FIG. 9 shows a gas chromatogram of a product obtained from the organic phase of Example 1 of the present application after distillation; wherein, 10.19′ corresponds to a compound of formula I, 12.20′ corresponds to a compound of formula IV-1, and 15.89′ and 16.08′ correspond to an impurity compound of formula III.

FIG. 10 shows a gas chromatogram of a product obtained from the organic phase of Example 2 of the present application after distillation; wherein, 10.62′ corresponds to a compound of formula I, and 15.53′ and 15.80′ correspond to an impurity compound of formula III.

FIG. 11 shows a gas chromatogram of a product obtained from the organic phase of Example 14 of the present application after distillation; wherein, 10.20′ corresponds to a compound of formula I.

FIG. 12 shows a gas chromatogram of a product obtained from the organic phase of Example 23 of the present application after distillation; wherein, 10.61′ corresponds to a compound of formula I, and 15.53′ and 15.80′ correspond to an impurity compound of formula III.

FIG. 13 shows a gas chromatogram of a product obtained from the organic phase of Example 1 of the present application after distillation; wherein, 10.17′ corresponds to a compound of Formula I, and 15.89′ and 16.07′ correspond to an impurity compound of formula III.

### DESCRIPTION OF THE EMBODIMENTS

In order to make the purpose, technical solutions and advantages of the present application clearer, the technical solutions in the examples of the present application will be described clearly and completely. Obviously, the described examples are some of the examples of the present application, but not all of them. Based on the examples of the present invention, all other examples obtained by those of ordinary skill in the art without creative work are falling within the protection scope of the present invention.

In addition, in order to better explain the present application, a lot of specific details are given in the following examples. It will be understood by those skilled in the art that the present application may be implemented without certain specific details. In some examples, materials, solutions, methods, means, etc., well known to those skilled in the art, are not described in detail so as to highlight the spirit of the present application.

Throughout the specification and claims, the term “comprising” or variations thereof, such as “including” or “containing”, will be understood to include the stated components and not to exclude other elements or other components, unless expressly indicated otherwise.

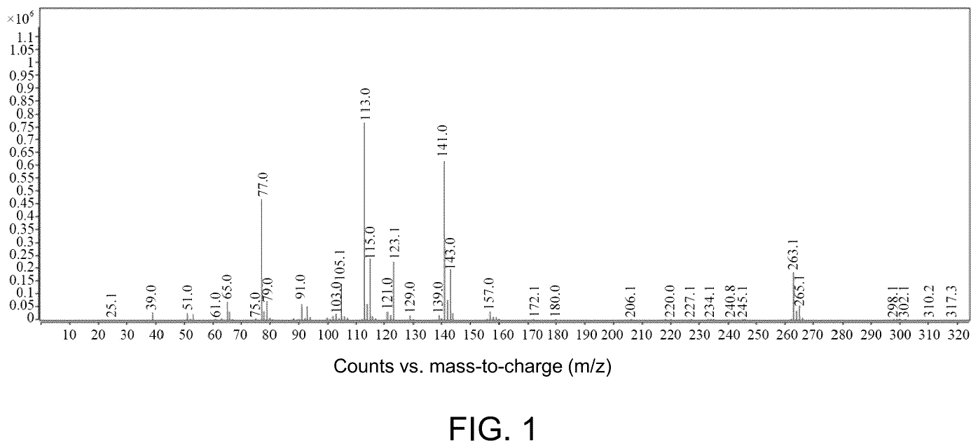

When repeating the methods reported in the prior art (CN1440376A, CN105693569A, Ge, Fa-xiang, Synthesis of post-emergence paddy herbicide benzob, Anhui Huagong (2013), 39(6), 41-43), the inventors have found that during extraction, not only flocs appear at the interface of the aqueous and oil phases, which may lead to layering difficulties (as shown in FIG. 8-a), but also an obvious ether impurity appears in the organic phase, which, as detected by GC-MS, has a molecular weight of 298. Based on the nature of formulas I and II and the mass spectral molecular weight GC-MS (EI) of 298.1 (the mass spectrum is shown in FIG. 1), it is speculated that the structure of M298 is 4,4′-oxybis(3-chlorobicyclo[3.2.1]oct-2-ene) as shown in formula III:

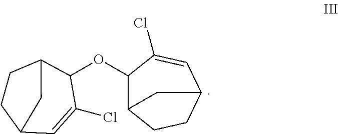 [SMILES: ClC1=CC2CCC(C2)C1OC1C(Cl)=CC2CCC1C2]

The boiling point of the impurity compound shown in formula III is higher than that of the target product (the compound of Formula I), and complex operations such as rectification are required for further purification, which greatly affects the production efficiency. For subsequent reactions to proceed normally, the purity of formula I needs to be greater than 94%. If the content of ether impurity (III) in the target product is high but without any purification, it will affect subsequent reactions, in the prior art route, about 5% of ether impurity (III) may be present in the product after extraction and distillation of the solvent, which require rectification and other complex purifications, thus greatly affecting the production efficiency.

In order to ensure the yield while minimizing the formation of impurity compounds (e.g., ether impurities (III)), the inventors have made various attempts, comprising:

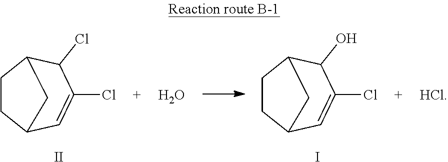

When the compound of formula II and water are stirred at room temperature, the resultant aqueous phase is acidic, and trace amounts of the compound of formula I are detected. It is further found that increasing the reaction temperature leads to a conversion up to 54%, a yield of 35%, and a reduced selectivity, as shown in Table 1:

TABLE 1
Reaction situations of the compound of formula
II undergoing direct hydrolysis in water

| | Reaction | Amount of | Reaction | Conversion | | |
|---|---|---|---|---|---|---|
| No. | temperature/° C. | water | time | (%) | Selectivity | Yield |
| 1 | 60 | 10x mass | 6 h | 23.4% | 73.3% | 17.2% |
| 2 | 75 | 10x mass | 6 h | 35.3% | 68.8% | 24.3% |
| 3 | 90 | 10x mass | 6 h | 53.5% | 65.7% | 35.1% |
| 4 | 100 | 10x mass | 6 h | 54.6% | 48.0% | 26.2% |

As seen from Table 1, the direct yield in water is poor and an impurity compound of formula III are still formed in Route B-1.

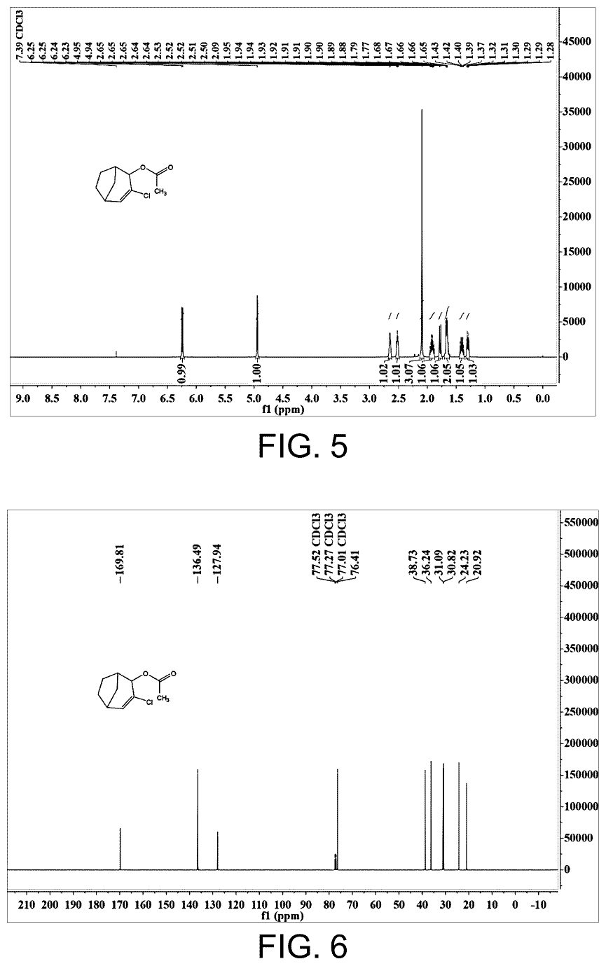

By further researching, the inventors have found that the addition of sodium acetate to the B-1 route may lead to the formation of 3-chlorobicyclo[3.2.1]-3-octen-2-acetate in the system, which has the following structure (NMR data are shown in FIGS. 5 and 6).

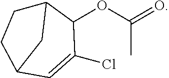

The inventors have found that the formation of 3-chlorobicyclo[3.2.1]-3-octen-2-acetate may result in a decrease in the formation of impurity—a compound of formula III. However, further hydrolysis is required for the conversation of the compound of formula IV to the compound of formula I.

The inventors have also tried various carboxylates—sodium formate, potassium acetate, sodium propionate, potassium propionate, sodium 4-chlorobenzoate, potassium 4-chlorobenzoate, sodium benzoate, potassium benzoate, etc., which also lead to the production of ester compounds, which have the structure shown in formula IV:

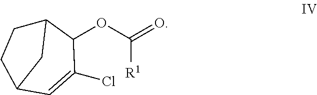

In the compound of formula IV, R¹ is hydrogen, alkyl, phenyl, substituted alkyl (preferably chloro-substituted alkyl) or substituted phenyl (preferably chloro-substituted phenyl); optionally, R¹ is hydrogen, methyl, ethyl, 4-chlorophenyl or phenyl.

In this way, the inventors obtain Reaction route B-2, which is carried out in water as a solvent:

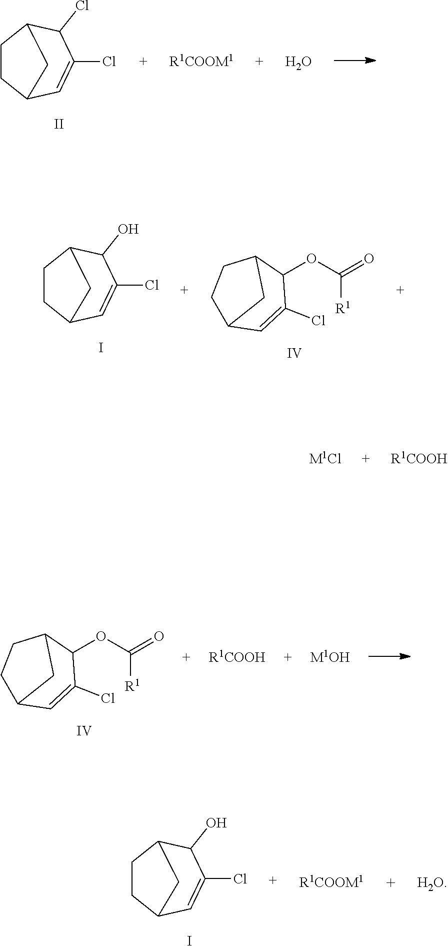

In Reaction route B-2, R¹ is hydrogen, alkyl, phenyl, substituted alkyl (preferably chloro-substituted alkyl) or substituted phenyl (preferably chloro-substituted phenyl); optionally, R¹ is hydrogen, methyl, ethyl, 4-chlorophenyl or phenyl.

In Reaction route B-2, M¹ is a metal, optionally an alkali metal or alkali earth metal, preferably one or more of potassium, sodium, calcium and magnesium.

In Reaction route B-2, the reaction is carried out in water in the presence of a salt of strong base and weak acid (a carboxylate, preferably an alkali metal or alkali earth metal carboxylate) to give the compounds of Formula I and Formula IV, and then the compound of Formula IV is hydrolyzed to the compound of Formula I by adding an alkali metal or alkali earth metal hydroxide at a reduced temperature, which effectively reduce the formation of polymers and impurities (for example, a compound shown in Formula III). The reaction can also be carried out by adding a mixture of a strong base weak acid and an alkali metal or alkali earth metal hydroxide, which, however, may result in a decrease in both purity and yield (see Examples 2 to 13).

In Reaction route B-2, in the first step of esterification, an alkali metal carboxylate or an alkali earth metal carboxylate is employed; preferably, the carboxylate is one or more selected from the group consisting of an alkali metal formate, an alkali metal acetate, an alkali metal propionate, an alkali metal 4-chlorobenzoate, an alkali metal benzoate, an alkali earth metal formate, an alkali earth metal acetate, an alkali earth metal propionate, an alkali earth metal 4-chlorobenzoate, and an alkali earth metal benzoate; or the carboxylate is one or more selected from the group consisting of sodium formate, sodium acetate, potassium acetate, calcium acetate, magnesium acetate, sodium propionate, potassium propionate, sodium 4-chlorobenzoate, potassium 4-chlorobenzoate, sodium benzoate, and potassium benzoate, preferably one or more selected from the group consisting of sodium acetate, potassium acetate and potassium propionate. In this step, the reaction temperature is 0° C. to a reflux temperature of each solvent, preferably, 50° C. to a reflux temperature of each solvent, and more preferably 90° C. to a reflux temperature of each solvent.

In Reaction route B-2, the inorganic strong base used for the second step of hydrolysis is one or more of an alkali metal or alkali earth metal hydroxide; optionally, the alkali metal hydroxide comprises one or more of sodium hydroxide, potassium hydroxide, and lithium hydroxide; optionally, the alkali earth metal hydroxide comprises one or more of calcium hydroxide and magnesium hydroxide; optionally, the inorganic strong base is selected from one or more of sodium hydroxide and potassium hydroxide.

In the preparation method of the route B-2, in the two-step reaction, further, a catalyst may be employed, which is selected from polyethylene glycol, tetrabutylammonium bromide, tetrabutylammonium chloride, benzyltrimethylammonium chloride, diisopropylethylamine, triethylamine, triethylene diamine, crown ether, and optionally selected from tetrabutylammonium bromide, benzyltrimethylammonium chloride, tetrabutylammonium chloride, and polyethylene glycol.

By further researching, the inventors have found that, in Reaction route B-2, the first step of esterification is carried out under an anhydrous condition, which prevents the formation of ether impurity—the compound of formula III, after filtration of the salt and distillation of the solvent, an aqueous solution of an alkali metal or alkali earth metal hydroxide is added for hydrolysis at room temperature without subjecting to a high temperature, resulting in basically free of impurity of formula III, and improved selectivity and yield (see Examples 14 to 22). That is Reaction route B-3, the first step therein is carried out under an anhydrous condition in an organic solvent, followed by a second step of hydrolysis:

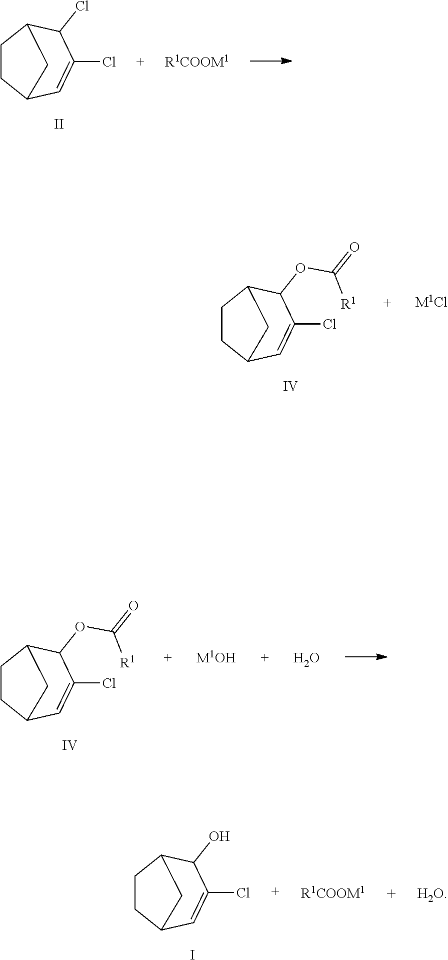

In Reaction route B-3, R¹ is hydrogen, alkyl, phenyl, substituted alkyl (preferably chloro-substituted alkyl) or substituted phenyl (preferably chloro-substituted phenyl); optionally, R¹ is hydrogen, methyl, ethyl, 4-chlorophenyl or phenyl.

In Reaction route B-3, M¹ is a metal, optionally an alkali metal or alkali earth metal, preferably one or more of potassium, sodium, calcium and magnesium.

In Reaction route B-3, the first step of esterification is carried out in an organic solvent, which can be one or more of dimethylformamide, dimethylacetamide, ethylene glycol monomethyl ether, ethylene glycol dimethyl ether, ethylene glycol diethyl ether, N-methyl pyrrolidone, dimethyl sulfoxide, and sulfolane; optionally can be one or more of dimethylformamide and dimethylacetamide.

In Reaction route B-3, in the first step of esterification, a catalyst is selected from polyethylene glycol, tetrabutylammonium bromide, tetrabutylammonium chloride, diisopropylethylamine, triethylamine, triethylene diamine, crown ether, and optionally selected from tetrabutylammonium bromide, benzyltrimethylammonium chloride, and polyethylene glycol.

In Reaction route B-3, in the second step of hydrolysis after esterification, all or part of the solvent is distilled out, followed by adding water and an inorganic strong base, wherein, the inorganic strong base is one or more of an alkali metal or alkali earth metal hydroxide; optionally, the alkali metal hydroxide comprises one or more of sodium hydroxide, potassium hydroxide, and lithium hydroxide; optionally, the alkali earth metal hydroxide comprises one or more of calcium hydroxide and magnesium hydroxide; optionally, the inorganic strong base is selected from one or more of sodium hydroxide and potassium hydroxide.

In Reaction route B-3, in the second step of hydrolysis, the catalyst is selected from polyethylene glycol, tetrabutylammonium bromide, tetrabutylammonium chloride, benzyltrimethylammonium chloride, diisopropylethylamine, triethylamine, triethylene diamine, crown ether, and optionally selected from tetrabutylammonium bromide, benzyltrimethylammonium chloride, tetrabutylammonium chloride, and polyethylene glycol.

Furthermore, the inventors have made attempts to design Reaction route B-4 as follows by adding an inorganic salt of strong base and weak acid in Reaction route B-1 for direct hydrolysis to formula I without esterification, meanwhile reducing the formation of impurity of formula III:

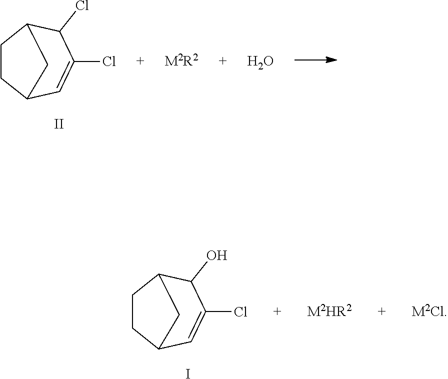

In Reaction route B-4, M² is a metal, optionally an alkali metal, preferably one or more of potassium and/or magnesium;

R² is an inorganic weak acid radical, optionally a carbonate radical, a phosphate radical, a hydrogen phosphate radical.

By employing Reaction route B-4, the formation of impurity of formula III can be reduced, the reaction rate is lowered, and basically no flocs are formed in layering during extraction. However, trace amounts of impurity of formula III are still formed in a slightly lower yield compared to Route B-3 (see Examples 23-32), and the distilled product may be used directly for subsequent reactions.

In the reaction of Reaction route B-4, the salt of strong base and weak acid is one or more selected from the group consisting of sodium carbonate, potassium carbonate, tripotassium phosphate, dipotassium hydrogen phosphate; optionally, the salt of strong base and weak acid comprises one or more of sodium carbonate and potassium carbonate.

In the reaction of Reaction route B-4, the catalyst is selected from polyethylene glycol, tetrabutylammonium bromide, tetrabutylammonium chloride, benzyltrimethylammonium chloride, diisopropylethylamine, triethylamine, and triethylene diamine, and optionally from tetrabutylammonium bromide, benzyltrimethylammonium chloride, tetrabutylammonium chloride, and polyethylene glycol.

In the present application, synthesis can be carried out by one way selected from Reaction routes B-2, B-3, and B-4, depending on the raw materials or solvent. Reaction routes B-2 and B-4 can achieve the following advantages by replacing the alkali type and lowering the alkali equivalent: reducing alkali waste and wastewater treatment difficulty; lowering the alkalinity of the reaction solution, thereby reducing the formation of polymers and impurities, making layering clear, and improving purification efficiency; and enabling the purity of product after distillation up to 94%, which can be used directly for subsequent reactions. The esterification reaction of Reaction route B-3 under an anhydrous condition is effective in avoiding impurity formation, and a low-temperature hydrolysis helps to prevent polymer formation. The inventive method can be carried out under simple and mild reaction conditions with high yield, which makes it suitable for industrial production; additionally, the purity of product after distillation can reach 94%, allowing for direct use in subsequent reactions.

The contents of the product in the following examples are determined by liquid or gas chromatography, in-process tracking during the reaction is carried out by area normalization. After the reaction ends, the purity of the product is determined by external standard calibration to calculate the yield.
LCMS: Liquid chromatography mass spectrometry.
GCMS: Gas chromatography mass spectrometry.
HPLC: High Performance Liquid Chromatography.
GC: Gas chromatography.
NMR: Nuclear magnetic resonance spectrometry.

3,4-dichlorobicyclo[3.2.1]-2-octene in the following examples can be obtained commercially or homemade; the reaction process and the results are detected by high-pressure liquid chromatography or gas chromatography, if not otherwise specified.

### Example 1

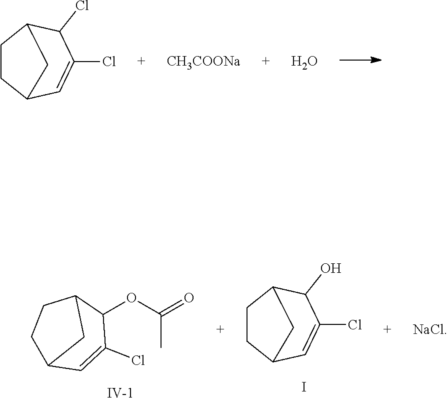

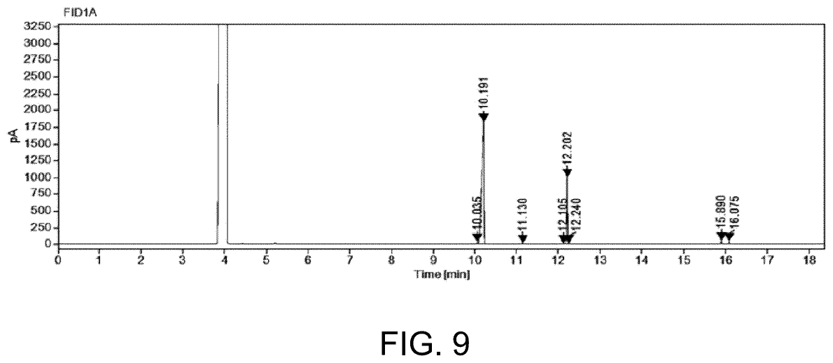

A mixture of 3,4-dichlorobicyclo[3.2.1]-2-octene (88.5 g, 0.5 mol), water (200 mL), sodium acetate (82.0 g, 1.0 mol) and tetrabutylammonium bromide (0.1 g) was heated to reflux for approximately 7 h, and the reaction was conducted under in-process control by GC until the amount of 3,4-dichlorobicyclo[3.2.1]-2-octene decreased to less than 1%, at which point the reaction was terminated. After cooling, the mixture was extracted with dichloroethane, and the resultant organic phase was dried with sodium sulfate and distilled to give 83.5 g of a yellow oil, the contents of which were determined by quantitative analysis as follows: 57.9 g of 3-chlorobicyclo[3.2.1]-3-octen-2-ol (I), 21.1 g of 3-chlorobicyclo[3.2.1]-3-octen-2-acetate (a compound of formula IV-1, which can be hydrolyzed to a compound of formula I), and the gas chromatogram of which was shown in FIG. 9, wherein ether impurity of formula III (15.89′ and 16.08′) accounted for 1.2% (by area normalization), the compound of formula I (10.19′) accounted for 80.4% (by area normalization), the compound of formula IV-1 (12.20′) accounted for 17.4% (by area normalization), with the sum of the compounds of formula IV-1 and formula I yielding 94%.

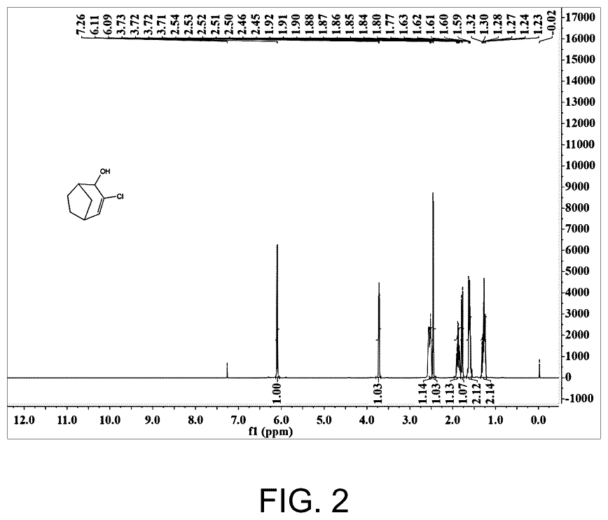

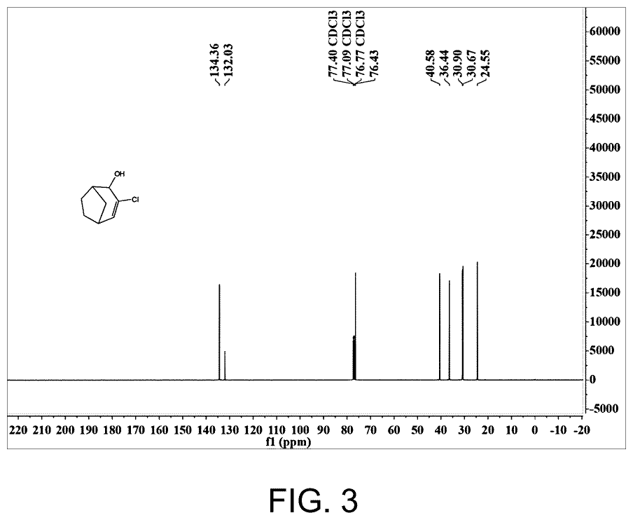

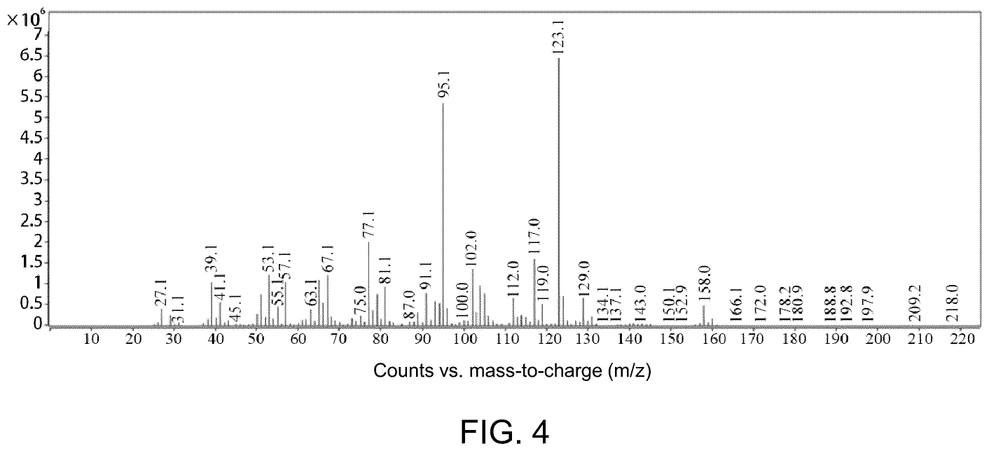

The NMR H and C spectra and GC-MS analysis (FIGS. 2, 3 and 4) of the intermediate 3-chlorobicyclo[3.2.1]-3-octen-2-ol (I) were analyzed as follows:

¹H-NMR (400 MHz, Chloroform-d) δ6.10 (d, J=7.1 Hz, 1H), 3.72 (dd, J=4.9, 2.9 Hz, 1H), 2.54˜2.48 (m, 1H), 2.46 (d, J=4.8 Hz, 1H), 1.88 (ddt, J=13.7, 9.6, 5.2 Hz, 1H), 1.78 (d, J=11.4 Hz, 1H), 1.68˜1.56 (m, 2H), 1.35˜1.21 (m, 2H).

¹³C-NMR (101 MHz, Chloroform-d) δ134.36, 132.03, 77.40, 77.09, 76.77, 76.43, 40.58, 36.44, 30.90, 30.67, 24.55.

GC-MS (EI): 158.0.

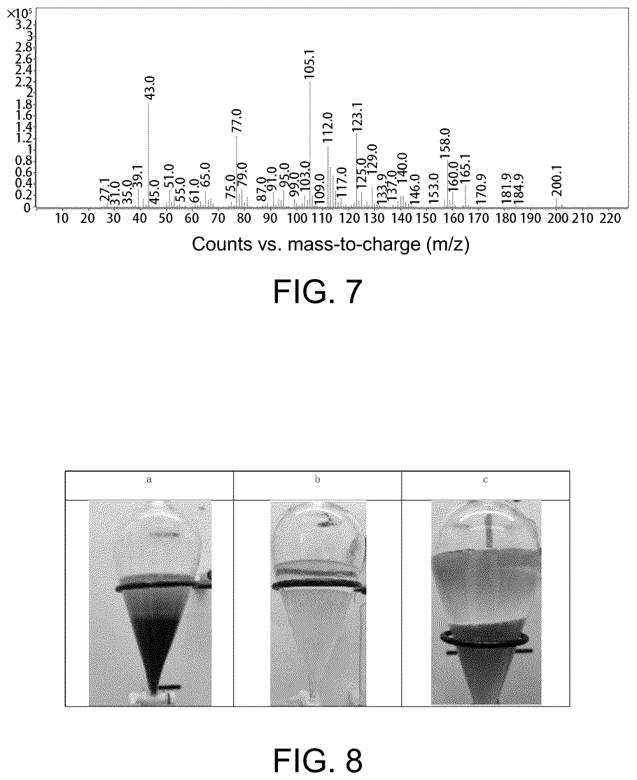

The NMR H and C spectra and GC-MS analysis (FIGS. 5, 6 and 7) of the intermediate 3-chlorobicyclo[3.2.1]-3-octen-2-acetate were analyzed as follows:

¹H-NMR (500 MHz, Chloroform-d) δ6.24 (dd, J=7.2, 1.0 Hz, 1H), 4.94 (d, J=3.0 Hz, 1H), 2.67˜2.62 (m, 1H), 2.52 (t, J=8.1 Hz, 1H), 2.09 (s, 3H), 1.97˜1.87 (m, 1H), 1.78 (d, J=9.7 Hz, 1H), 1.70˜1.62 (m, 2H), 1.45˜1.36 (m, 1H), 1.30 (m, 1H).

¹³C-NMR (126 MHz, Chloroform-d) δ169.81, 136.49, 127.94, 76.41, 38.73, 36.24, 31.09, 30.82, 24.23, 20.92.

GC-MS (EI): 200.1.

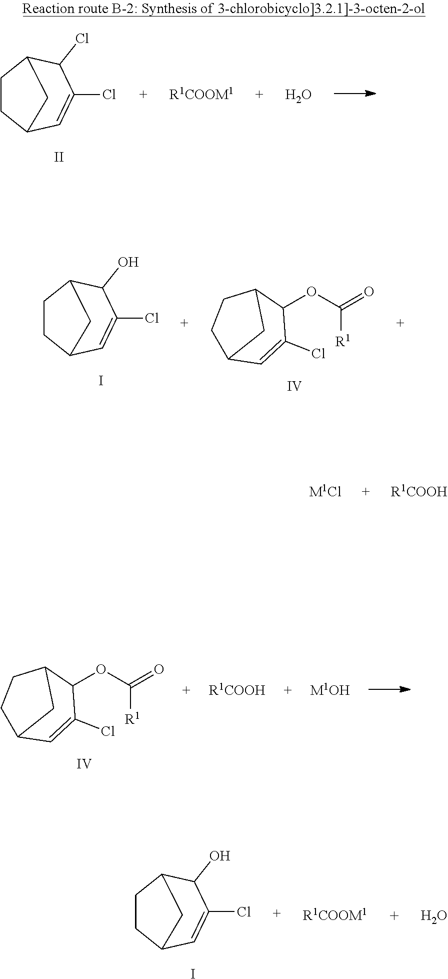

### Example 2

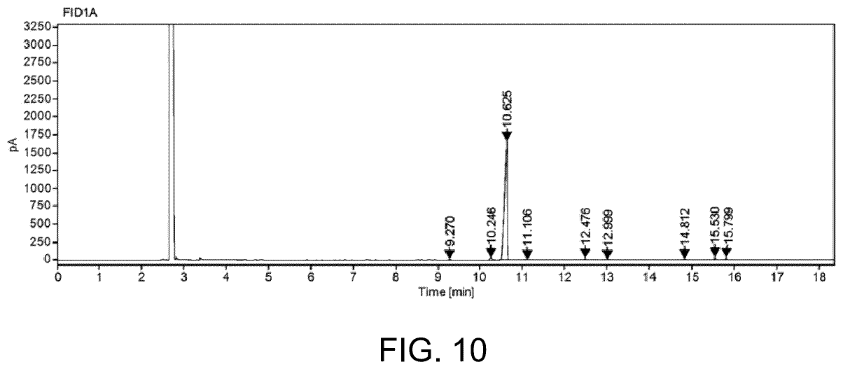

A mixture of 3,4-dichlorobicyclo[3.2.1]-2-octene (88.5 g, 0.5 mol), water (200 mL), sodium acetate (82.0 g, 1.0 mol) and tetrabutylammonium bromide (0.1 g) was heated to a reflux temperature and kept for about 6 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 1 h. After cooling, the mixture was extracted with ethylene dichloride without any floc formation during layering (as shown in FIG. 8-b), and the resultant organic phase was dried with sodium sulfate and distilled to give 78.4 g of a yellow oil. As shown in the gas chromatogram of FIG. 10, ether impurity of formula III (15.53′ and 15.80′) accounted for 1.3% (by area normalization), and the compound of formula I (10.625′) accounted for 96.2% (by area normalization). The purity of the target product (the compound of formula I) was 95.3% (by external standard calibration), and the yield was 94.2%.

### Example 3

A mixture of 3,4-dichlorobicyclo[3.2.1]-2-octene (88.5 g, 0.5 mol), water (200 mL), sodium acetate (82.0 g, 1.0 mol) and tetrabutylammonium bromide (0.1 g) was heated to 90° C. and kept for about 8 h. After cooling to 50° C., sodium hydroxide (20.0 g, 0.5 mol) was added, and the reaction was kept at the temperature for 1 h. After cooling, the mixture was extracted with ethylene dichloride without any floc formation during layering, and the resultant organic phase was dried with sodium sulfate and distilled to give 78.3 g of a yellow oil. As shown in the gas chromatogram, only trace amounts of impurity compound of formula III were formed, the purity of the target product (the compound of formula I) was 94.9% (by external standard calibration), and the yield was 93.7%.

### Example 4

A mixture of 3,4-dichlorobicyclo[3.2.1]-2-octene (88.5 g, 0.5 mol), water (200 mL), sodium acetate (82.0 g, 1.0 mol) and tetrabutylammonium bromide (0.1 g) was heated to 103° C. for refluxing for 5 h. After cooling to 20° C., sodium hydroxide (20.0 g, 0.5 mol) was added, and the reaction was kept at the temperature for 3 h. After cooling, the mixture was extracted with ethylene dichloride without any floc formation during layering, the resultant organic phase was dried with sodium sulfate and distilled to give 78.9 g of a yellow oil. As shown in the gas chromatogram, only trace amounts of impurity compound of formula III were formed, the purity of the target product (the compound of formula I) was 95.5% (by external standard calibration), and the yield was 95.0%.

### Example 5A

A mixture of 3,4-dichlorobicyclo[3.2.1]-2-octene (88.5 g, 0.5 mol), water (200 mL), sodium acetate (82.0 g, 1.0 mol) and tetrabutylammonium bromide (0.1 g) was heated to 103° C. for refluxing for 5 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 1 h. After cooling, the mixture was extracted with ethylene dichloride without any floc formation during layering, and the resultant organic phase was dried with sodium sulfate and distilled to give 78.6 g of a yellow oil. As shown in the gas chromatogram, only trace amounts of impurity compound of formula III were formed, the purity of the target product (the compound of formula I) was 95.4% (by external standard calibration), and the yield was 94.5%.

### Example 5B

A mixture of 3,4-dichlorobicyclo[3.2.1]-2-octene (88.5 g, 0.5 mol), water (200 mL), and sodium acetate (82.0 g, 1.0 mol) was heated to 103° C. for refluxing for 9 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 3 h. After cooling, the mixture was extracted with ethylene dichloride without any floc formation during layering, and the resultant organic phase was dried with sodium sulfate and distilled to give 78.1 g of a yellow oil. As shown in the gas chromatogram, only trace amounts of impurity compound of formula III were formed, the purity of the target product (the compound of formula I) was 94.6% (by external standard calibration), and the yield was 93.2%.

### Example 5C

A mixture of 3,4-dichlorobicyclo[3.2.1]-2-octene (88.5 g, 0.5 mol), water (200 mL), sodium acetate (82.0 g, 1.0 mol) and tetrabutylammonium bromide (5.0 g) was heated to 103° C. for refluxing for 4 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 1 h After cooling, the mixture was extracted with ethylene dichloride without any floc formation during layering, and the resultant organic phase was dried with sodium sulfate and distilled to give 78.9 g of a yellow oil. As shown in the gas chromatogram, only trace amounts of impurity compound of formula III were formed, the purity of the target product (the compound of formula I) was 95.5% (by external standard calibration), and the yield was 95.0%.

### Example 6

A mixture of 3,4-dichlorobicyclo[3.2.1]-2-octene (88.5 g, 0.5 mol), water (200 mL), sodium acetate (82.0 g, 1.0 mol) and tetrabutylammonium bromide (0.1 g) was heated to 103° C. for refluxing for 5 h, and the reaction was conducted under in-process control by GC until the amount of 3,4-dichlorobicyclo[3.2.1]-2-octene decreased to less than 1%, at which point the reaction was terminated. After cooling to 40° C., sodium hydroxide (20.0 g, 0.5 mol) was added, and the reaction was kept at the temperature for 2 h. After cooling, the mixture was extracted with ethylene dichloride without any floc formation during layering, and the resultant organic phase was dried with sodium sulfate and distilled to give 78.6 g of a yellow oil. As shown in the gas chromatogram, only trace amounts of impurity compound of formula III were formed, the purity of the target product (the compound of formula I) was 95.2% (by external standard calibration), and the yield was 94.4%.

### Example 7

A mixture of 3,4-dichlorobicyclo[3.2.1]-2-octene (88.5 g, 0.5 mol), water (200 mL), sodium acetate (69.7 g, 0.85 mol) and tetrabutylammonium bromide (0.1 g) was heated to 103° C. for refluxing for 5 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (40.0 g, 1.0 mol) was added, and the reaction was kept at the temperature for 1 h. After cooling, the mixture was extracted with ethylene dichloride without any floc formation during layering, and the resultant organic phase was dried with sodium sulfate and distilled to give 78.1 g of a yellow oil. As shown in the gas chromatogram, only trace amounts of impurity compound of formula III were formed, the purity of the target product (the compound of formula I) was 95.1% (by external standard calibration), and the yield was 93.7%.

### Example 8

A mixture of 3,4-dichlorobicyclo[3.2.1]-2-octene (88.5 g, 0.5 mol), water (150 mL), sodium acetate (61.5 g, 0.75 mol) and tetrabutylammonium bromide (0.1 g) was heated to 103° C. for refluxing for 5 h. After cooling to 50° C., sodium hydroxide (20.0 g, 0.5 mol) was added, and the reaction was kept at the temperature for 1 h. After cooling, the mixture was extracted with ethylene dichloride without any floc formation during layering, and the resultant organic phase was dried with sodium sulfate and distilled to give 76.2 g of a yellow oil. As shown in the gas chromatogram, only trace amounts of impurity compound of formula III were formed, the purity of the target product (the compound of formula I) was 95.0% (by external standard calibration), and the yield was 91.3%.

### Example 9A

A mixture of 3,4-dichlorobicyclo[3.2.1]-2-octene (88.5 g, 0.5 mol), water (290 mL), sodium acetate (123.0 g, 1.5 mol) and tetrabutylammonium bromide (0.1 g) was heated to 103° C. for refluxing for 5 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 1 h. After cooling, the mixture was extracted with ethylene dichloride without any floc formation during layering, and the resultant organic phase was dried with sodium sulfate and distilled to give 79.1 g of a yellow oil. As shown in the gas chromatogram, only trace amounts of ether impurity of formula III were formed, the purity of the target product (the compound of formula I) was 95.2% (by external standard calibration), and the yield was 95.0%.

### Example 9B

A mixture of 3,4-dichlorobicyclo[3.2.1]-2-octene (88.5 g, 0.5 mol), water (900 mL), sodium acetate (123.0 g, 1.5 mol) and tetrabutylammonium bromide (0.1 g) was heated to 103° C. for refluxing for 6 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 2 h. After cooling, the mixture was extracted with ethylene dichloride without any floc formation during layering, and the resultant organic phase was dried with sodium sulfate and distilled to give 78.8 g of a yellow oil. As shown in the gas chromatogram, only trace amounts of impurity compound of formula III were formed, the purity of the target product (the compound of formula I) was 95.4% (by external standard calibration), and the yield was 94.8%.

### Example 10

A mixture of 3,4-dichlorobicyclo[3.2.1]-2-octene (88.5 g, 0.5 mol), water (470 mL), sodium acetate (82.0 g, 1.0 mol) and tetrabutylammonium bromide (0.1 g) was heated to 103° C. for refluxing for 5 h, and the reaction was conducted under in-process control by GC until the amount of 3,4-dichlorobicyclo[3.2.1]-2-octene decreased to less than 1%, at which point the reaction was terminated. After cooling to 50° C., sodium hydroxide (20.0 g, 0.5 mol) was added, and the reaction was kept at the temperature for 2 h. After cooling, the mixture was extracted with ethylene dichloride without any floc formation during layering, and the resultant organic phase was dried with sodium sulfate and distilled to give 78.2 g of a yellow oil. As shown in the gas chromatogram, only trace amounts of impurity compound of formula III were formed, the purity of the target product (the compound of formula I) was 95.4% (by external standard calibration), and the yield was 94.0%.

### Example 11

A mixture of 3,4-dichlorobicyclo[3.2.1]-2-octene (88.5 g, 0.5 mol), water (200 mL), sodium acetate (82.0 g, 1.0 mol), sodium hydroxide (20.0 g, 0.5 mol) and tetrabutylammonium bromide (0.1 g) was heated to 103° C. for refluxing for 6 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride, resulting in trace amounts of flocs during layering, and the resultant organic phase was dried with sodium sulfate and distilled to give 78.0 g of a yellow oil. As shown in the gas chromatogram, impurity compound of formula III accounted for 2.2% (by area normalization), the purity of the target product (the compound of formula I) accounted for 94.1% (by external standard calibration), and the yield was 92.5%.

In this example, by adding sodium acetate and sodium hydroxide at the same time, sodium acetate will be reacted with the compound of formula II to form an ester, while decreasing the usage of sodium hydroxide, thereby reducing the chance of ether formation. Although trace amounts of impurity of formula III will be formed, which are less than that of the prior art. However, due to such simultaneous addition, trace amounts of flocs and impurity compound of formula III will also be formed, resulting in a decrease in both yield and purity of the product.

### Example 12

A mixture of 3,4-dichlorobicyclo[3.2.1]-2-octene (88.5 g, 0.5 mol), water (200 mL), sodium acetate (82.0 g, 1.0 mol), potassium hydroxide (28.1 g, 0.5 mol) and tetrabutylammonium bromide (0.1 g) was heated to 103° C. for refluxing for 6 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride, resulting in trace amounts of flocs during layering, and the resultant organic phase was dried with sodium sulfate and distilled to give 78.0 g of a yellow oil. As shown in the gas chromatogram, impurity compound of formula III accounted for 2.3% (by area normalization), the purity of the target product (the compound of formula I) accounted for 94.3% (by external standard calibration), and the yield was 92.7%.

By adding sodium acetate and potassium hydroxide at the same time, sodium acetate will be reacted with the compound of formula II to form an ester, thereby reducing the chance of ether formation. Although trace amounts of impurity of formula III will be formed, which are less than that of the prior art.

### Example 13

A mixture of 3,4-dichlorobicyclo[3.2.1]-2-octene (88.5 g, 0.5 mol), water (200 mL), sodium acetate (82.0 g, 1.0 mol), sodium hydroxide (20.0 g, 0.5 mol) and benzyltrimethylammonium chloride (0.1 g) was heated to 103° C. for refluxing for 6 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride, resulting in trace amounts of flocs during layering, and the resultant organic phase was dried with sodium sulfate and distilled to give 78.6 g of a yellow oil. As shown in the gas chromatogram, impurity compound of formula III accounted for 2.1% (by area normalization), the purity of the target product (the compound of formula I) accounted for 94.2% (by external standard calibration), and the yield was 93.3%.

By adding sodium acetate and sodium hydroxide at the same time, sodium acetate will be reacted with the compound of formula II to form an ester, thereby reducing the chance of ether formation. Although trace amounts of impurity of formula III will be formed, which are less than that of the prior art.

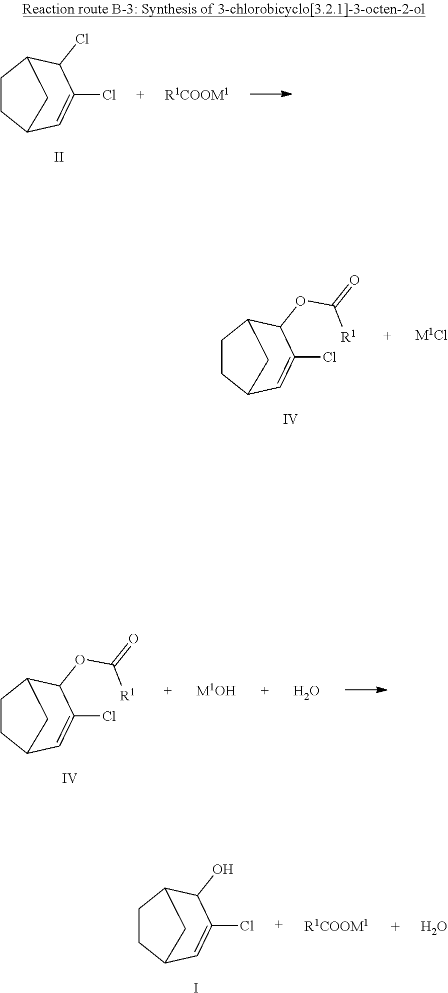

The yield in Reaction route B-3 will be improved under an anhydrous condition, without the formation of impurity compound of formula III nearly. In the following examples, although the purity was about 95% due to a small amount of solvent remaining after distillation, further distillation was not pursued to achieve higher purity since the presence of the solvent does not affect subsequent reactions.

### Example 14

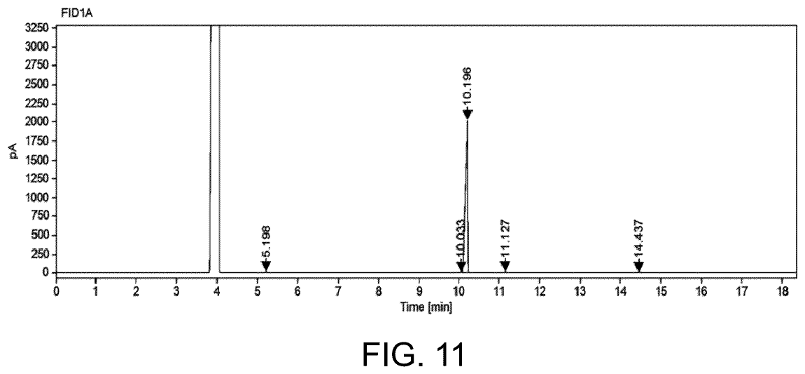

A mixture of 3,4-dichlorobicyclo[3.2.1]-2-octene (88.5 g, 0.5 mol), dimethylformamide (450 mL), anhydrous sodium acetate (82.0 g, 1.0 mol) and tetrabutylammonium bromide (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and tetrabutylammonium bromide (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 79.7 g of a yellow oil. As shown in the gas chromatogram of FIG. 11, no impurity compound of formula III appeared, the compound of formula I (10.20′) accounted for 98.5% (by external standard calibration), the purity of the target product (the compound of formula I) was 95.3% (by external standard calibration), and the yield was 95.8%.

### Example 15

A mixture of 3,4-dichlorobicyclo[3.2.1]-2-octene (88.5 g, 0.5 mol), dimethylacetamide (450 mL), anhydrous sodium acetate (82.0 g, 1.0 mol) and tetrabutylammonium bromide (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and tetrabutylammonium bromide (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 79.8 g of a yellow oil. As shown in the gas chromatogram, no impurity compound of formula III appeared, the purity of the target product (the compound of formula I) was 95.2% (by external standard calibration), and the yield was 95.8%.

### Example 16

A mixture of 3,4-dichlorobicyclo[3.2.1]-2-octene (88.5 g, 0.5 mol), ethylene glycol monomethyl ether (450 mL), anhydrous sodium acetate (82.0 g, 1.0 mol) and tetrabutylammonium bromide (0.1 g) was heated to reflux for 6 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and tetrabutylammonium bromide (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 77.8 g of a yellow oil. As shown in the gas chromatogram, no impurity compound of formula III appeared, the purity of the target product (the compound of formula I) was 95.0% (by external standard calibration), and the yield was 93.2%.

### Example 17

A mixture of 3,4-dichlorobicyclo[3.2.1]-2-octene (88.5 g, 0.5 mol), dimethylformamide (450 mL), anhydrous potassium acetate (98.1 g, 1.0 mol) and tetrabutylammonium bromide (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and tetrabutylammonium bromide (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 78.8 g of a yellow oil. As shown in the gas chromatogram, no impurity compound of formula III appeared, the purity of the target product (the compound of formula I) was 95.5% (by external standard calibration), and the yield was 94.9%.

### Example 18A

A mixture of 3,4-dichlorobicyclo[3.2.1]-2-octene (88.5 g, 0.5 mol), dimethylformamide (450 mL), anhydrous sodium acetate (123 g, 1.5 mol) and polyethylene glycol (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and benzyltrimethylammonium chloride (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 79.7 g of a yellow oil. As shown in the gas chromatogram, no impurity compound of formula III appeared, the purity of the target product (the compound of formula I) was 95.1% (by external standard calibration), and the yield was 95.6%.

### Example 18B

A mixture of 3,4-dichlorobicyclo[3.2.1]-2-octene (88.5 g, 0.5 mol), dimethylformamide (900 mL), anhydrous sodium acetate (123 g, 1.5 mol) and polyethylene glycol (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and benzyltrimethylammonium chloride (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 79.5 g of a yellow oil. As shown in the gas chromatogram, no impurity compound of formula III appeared, the purity of the target product (the compound of formula I) was 95.3% (by external standard calibration), and the yield was 95.5%.

### Example 19

A mixture of 3,4-dichlorobicyclo[3.2.1]-2-octene (88.5 g, 0.5 mol), dimethylformamide (450 mL), anhydrous sodium acetate (102.5 g, 1.25 mol) and polyethylene glycol (0.1 g) was heated to reflux for 6 h, then the reaction was terminated. The mixture was cooled down, filtered, and distilled to remove the solvent, followed by adding sodium hydroxide (30 g, 0.75 mol), water (270 ml) and benzyltrimethylammonium chloride (0.1 g), and stirring at 30° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 78.9 g of a yellow oil. As shown in the gas chromatogram, no impurity compound of formula III appeared, the purity of the target product (the compound of formula I) was 95.4% (by external standard calibration), and the yield was 94.9%.

### Example 20

A mixture of 3,4-dichlorobicyclo[3.2.1]-2-octene (88.5 g, 0.5 mol), dimethylformamide (450 mL), anhydrous sodium acetate (82.0 g, 1.0 mol) and tetrabutylammonium bromide (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and tetrabutylammonium bromide (0.1 g), and stirring at 50° C. for 2 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 79.5 g of a yellow oil. As in the gas chromatogram, no impurity compound of formula III appeared, the purity of the target product (the compound of formula I) was 95.0% (by external standard calibration), and the yield was 95.2%.

### Example 21

A mixture of 3,4-dichlorobicyclo[3.2.1]-2-octene (88.5 g, 0.5 mol), dimethylformamide (450 mL), anhydrous sodium acetate (82.0 g, 1.0 mol) and tetrabutylammonium bromide (0.1 g) was heated to reflux for 4 h, and the reaction was conducted under in-process control by GC until the amount of 3,4-dichlorobicyclo[3.2.1]-2-octene decreased to less than 1%, at which point the reaction was terminated. The mixture was cooled down, filtered, and distilled to remove the solvent, followed by adding sodium hydroxide (30 g, 0.75 mol), water (270 ml) and tetrabutylammonium bromide (0.1 g), and stirring at 60° C. for 1 h, and the reaction was conducted under in-process control by GC until the amount of 3-chlorobicyclo[3.2.1]-3-octen-2-acetate decreased to less than 0.5%, at which point the reaction was terminated. The reaction solution was extracted with ethylene dichloride without any floc formation during layering, and the resultant organic phase was dried with sodium sulfate and distilled to give 79.1 g of a yellow oil. As shown in the gas chromatogram, no impurity compound of formula III appeared, the purity of the target product (the compound of formula I) was 94.9% (by external standard calibration), and the yield was 94.7%.

### Example 22

A mixture of 3,4-dichlorobicyclo[3.2.1]-2-octene (88.5 g, 0.5 mol), dimethylformamide (450 mL), anhydrous sodium formate (68.0 g, 1.0 mol) and polyethylene glycol (0.1 g) was heated to reflux for 6 h, then the reaction was terminated. The mixture was cooled down, filtered, and distilled to remove the solvent, followed by adding sodium hydroxide (30 g, 0.75 mol), water (270 ml) and benzyltrimethylammonium chloride (0.1 g), and stirring at 30° C. for 2 h, and the reaction was conducted under in-process control by GC until the amount of 3-chlorobicyclo[3.2.1]-3-octen-2-formate decreased to less than 0.5%, at which point the reaction was terminated. The reaction solution was extracted with ethylene dichloride without any floc formation during layering, and the resultant organic phase was dried with sodium sulfate and distilled to give 78.7 g of a yellow oil. As shown in the gas chromatogram, the purity of the target product (the compound of formula I) was 95.1% (by external standard calibration), and the yield was 94.4%.

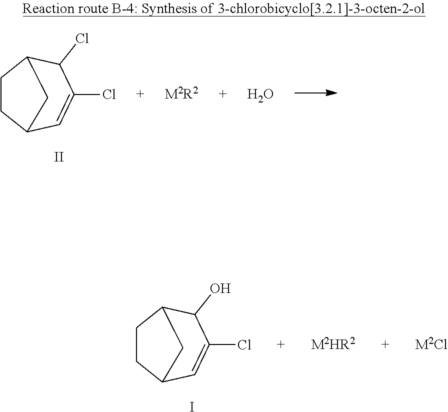

### Example 23

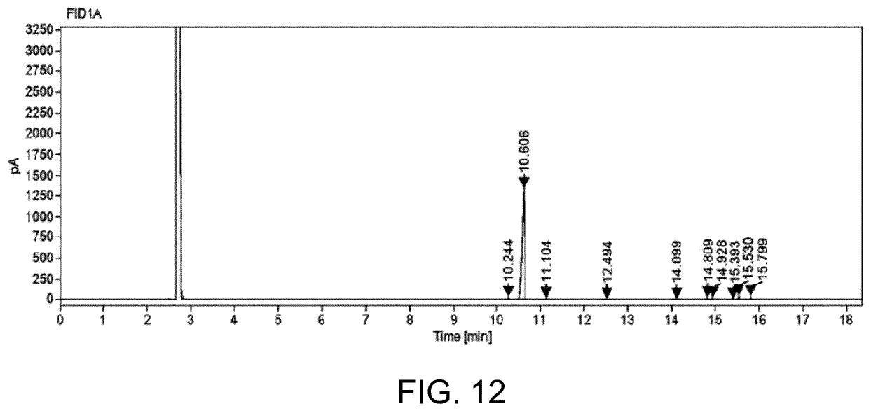

A mixture of 3,4-dichlorobicyclo[3.2.1]-2-octene (88.5 g, 0.5 mol), water (450 mL), sodium carbonate (79.5 g, 0.75 mol) and tetrabutylammonium bromide (0.1 g) was heated to 103° C. for refluxing for 7 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, as shown in FIG. 8-c, and the resultant organic phase was dried with sodium sulfate and distilled to give 76.3 g of a yellow oil. As shown in the gas chromatogram of FIG. 12, impurity compound of formula III (15.53′ and 15.80′) accounted for 1.9% (by external standard calibration), the compound of formula I (10.61′) accounted for 95.3% (by external standard calibration), the purity of the target product (the compound of formula I) was 95.1% (by external standard calibration), and the yield was 91.5%.

### Example 24

A mixture of 3,4-dichlorobicyclo[3.2.1]-2-octene (88.5 g, 0.5 mol), water (450 mL), sodium carbonate (79.5 g, 0.75 mol) and tetrabutylammonium bromide (0.1 g) was heated to 90° C. for 7 h, then the reaction was terminated. After cooling, the mixture was extracted with ethylene dichloride without any floc formation during layering, and the resultant organic phase was dried with sodium sulfate and distilled to give 76.9 g of a yellow oil. As shown in the gas chromatogram, only trace amounts of impurity compound of formula III appeared, the purity of the target product (the compound of formula I) was 94.8% (by external standard calibration), and the yield was 91.9%.

### Example 25

A mixture of 3,4-dichlorobicyclo[3.2.1]-2-octene (88.5 g, 0.5 mol), water (720 mL), sodium carbonate (79.5 g, 0.75 mol) and tetrabutylammonium bromide (0.1 g) was heated to 103° C. for refluxing for 7 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, and the resultant organic phase was dried with sodium sulfate and distilled to give 77.1 g of a yellow oil. As shown in the gas chromatogram, only trace amounts of impurity compound of formula III occurred, the purity of the target product (the compound of formula I) was 94.5% (by external standard calibration), and the yield was 91.9%.

### Example 26

A mixture of 3,4-dichlorobicyclo[3.2.1]-2-octene (88.5 g, 0.5 mol), water (1500 mL), sodium carbonate (79.5 g, 0.75 mol) and tetrabutylammonium bromide (0.1 g) was heated to 103° C. for refluxing for 8 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, and the resultant organic phase was dried with sodium sulfate and distilled to give 76.8 g of a yellow oil. As shown in the gas chromatogram, only trace amounts of impurity compound of formula III appeared, the purity of the target product (the compound of formula I) was 94.1% (by external standard calibration), and the yield was 91.1%.

### Example 27

A mixture of 3,4-dichlorobicyclo[3.2.1]-2-octene (88.5 g, 0.5 mol), water (300 mL), sodium carbonate (106 g, 1 mol) and tetrabutylammonium bromide (0.1 g) was heated to 103° C. for about 6 h, then the reaction was terminated. After cooling, the mixture was extracted with ethylene dichloride without any floc formation during layering, and the resultant organic phase was dried with sodium sulfate and distilled to give 77.0 g of a yellow oil. As shown in the gas chromatogram, only trace amounts of impurity compound of formula III appeared, the purity of the target product (the compound of formula I) was 94.9% (by external standard calibration), and the yield was 92.1%.

### Example 28

A mixture of 3,4-dichlorobicyclo[3.2.1]-2-octene (88.5 g, 0.5 mol), water (900 mL), sodium carbonate (159 g, 1.5 mol) and tetrabutylammonium bromide (0.1 g) was heated to 103° C. for refluxing for 7 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, and the resultant organic phase was dried with sodium sulfate and distilled to give 76.8 g of a yellow oil. As shown in the gas chromatogram, only trace amounts of impurity compound of formula III appeared, the purity of the target product (the compound of formula I) was 95.0% (by external standard calibration), and the yield was 92.0%.

### Example 29

A mixture of 3,4-dichlorobicyclo[3.2.1]-2-octene (88.5 g, 0.5 mol), water (650 mL), tripotassium phosphate (159.2 g, 0.75 mol) and tetrabutylammonium bromide (0.1 g) was heated to 103° C. for refluxing for 8 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, and the resultant organic phase was dried with sodium sulfate and distilled to give 77.6 g of a yellow oil. As shown in the gas chromatogram, only trace amounts of impurity compound of formula III appeared, the purity of the target product (the compound of formula I) was 95.2% (by external standard calibration), and the yield was 93.1%.

### Example 30

A mixture of 3,4-dichlorobicyclo[3.2.1]-2-octene (88.5 g, 0.5 mol), water (700 mL), dipotassium hydrogen phosphate (174.2 g, 1.0 mol) and tetrabutylammonium bromide (0.1 g) was heated to 103° C. for refluxing for 8 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, and the resultant organic phase was dried with sodium sulfate and distilled to give 77.4 g of a yellow oil. As shown in the gas chromatogram, only trace amounts of impurity compound of formula III appeared, the purity of the target product (the compound of formula I) was 94.8% (by external standard calibration), and the yield was 92.5%.

### Example 31

A mixture of 3,4-dichlorobicyclo[3.2.1]-2-octene (88.5 g, 0.5 mol), water (600 mL), potassium carbonate (103.7 g, 0.75 mol) and tetrabutylammonium bromide (0.1 g) was heated to 103° C. for refluxing for 7 h, and the reaction was conducted under in-process control by GC until the amount of 3,4-dichlorobicyclo[3.2.1]-2-octene decreased to less than 1%, at which point the reaction was terminated. After cooling, the mixture was extracted with ethylene dichloride without any floc formation during layering, and the resultant organic phase was dried with sodium sulfate and distilled to give 77.0 g of a yellow oil. As shown in the gas chromatogram, only trace amounts of impurity compound of formula III appeared, the purity of the target product (the compound of formula I) was 94.6% (by external standard calibration), and the yield was 91.8%.

### Example 32

A mixture of 3,4-dichlorobicyclo[3.2.1]-2-octene (88.5 g, 0.5 mol), water (450 mL), sodium carbonate (79.5 g, 0.75 mol) and polyethylene glycol (0.1 g) was heated to 103° C. for refluxing for 7 h. After cooling, the mixture was extracted with ethylene dichloride without any floc formation during layering, and the resultant organic phase was dried with sodium sulfate and distilled to give 76.9 g of a yellow oil. As shown in the gas chromatogram, only trace amounts of impurity compound of formula III appeared, the purity of the target product (the compound of formula I) was 94.7% (by external standard calibration), and the yield was 91.8%.

The purity of the products in the above examples can reach 94% or more after distillation with a small amount of solvent remaining therein, all of which can be directly used for subsequent reactions.

### Comparative Example Route A-1: Hydrolysis of 3-chlorobicyclo[3.2.1]-3-octen-2-ol

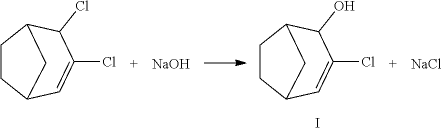

### Comparative Example 1

A mixture of 3,4-dichlorobicyclo[3.2.1]-2-octene (88.5 g, 0.5 mol), water (800 ml), sodium hydroxide (80.0 g, 2.0 mol), and benzyltrimethylammonium chloride (0.1 g) was heated to a reflux temperature and kept for 7 h. After cooling, the mixture was extracted with ethylene dichloride with flocs formation during layering as shown in FIG. 8-a, and the resultant organic phase was dried with sodium sulfate and distilled to give an orange brown oil, which was repeated three times, the data of the determined purity and the yield were shown in Table-2:

TABLE 2

| | Temperature/ | n (II:sodium hydroxide | | |
|---|---|---|---|---|
| No. | ° C. | solution) | Purity | Yield |
| 1 | Reflux | 1:4 | 89.1% | 88.7% |
| 2 | Reflux | 1:4 | 88.7% | 87.9% |
| 3 | Reflux | 1:4 | 89.3% | 89.2% |

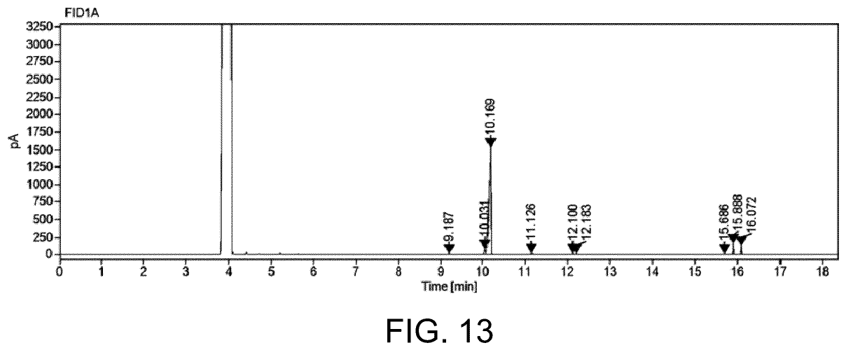

The presence of black tar and flocs in Comparative example 1 (as shown in FIG. 8-a) is likely due to the excessive dosage of alkali, resulting in strong alkalinity. Under these reaction conditions, it becomes easier to form more black tar and flocs, leading to a decrease in yield and purity. The product obtained from drying and distillation of the organic phase is shown in FIG. 13, where a compound of formula I (10.17′) accounts for 90.3% (by area normalization), while an impurity compound of formula III (15.89′ and 16.07′) accounts for 5.8% (by area normalization). The products obtained by this method require complex operations such as rectification for subsequent production, which greatly affects production efficiency.

### Comparative Example 2

According to the operation of Comparative example 1, a mixture of 3,4-dichlorobicyclo[3.2.1]-2-octene with different masses of water, sodium hydroxide and benzyltrimethylammonium chloride was heated to and kept at a temperature between 85° C. and the reflux temperature. After cooling, the mixture was extracted with ethylene dichloride with flocs formation during layering, and the resultant organic phase was dried with sodium sulfate and distilled to give an orange brown oil, which was repeated four times, the data of the determined purity and the calculated yield were shown in Table-3:

TABLE 3

| | | n (II:sodium hydroxide | | |
|---|---|---|---|---|
| No. | Temperature/° C. | solution) | Purity | Yield |
| 1 | 95 | 1:4 | 89.5% | 88.6% |
| 2 | 85 | 1:4 | 83.4% | 75.2% |
| 3 | 103 | 1:2 | 86.8% | 81.6% |
| 4 | 103 | 1:5 | 88.6% | 88.7% |

In Comparative examples 1 and 2, despite many adjustments to the conditions, a significant consumption of strong base in the synthesis of 3-chlorobicyclo[3.2.1]-3-octen-2-ol readily results in the formation of black tar, flocs (insoluble in water and dichloromethane), as well as impurities such as the compound of formula III, with the content of ether impurity being greater than 5%, leading to a decrease in yield and purity; meanwhile, the higher boiling point of ether impurity (formula III) compared to the compound of formula I makes distilling the product by rectification both energy-consuming and costly, and additionally, these remaining ether impurity (formula III) will dissolve the product, further reducing its yield and negatively impacting subsequent reactions without purification.

Finally, it should be noted that the above examples are only used to illustrate the technical solutions of the present application and not to limit it; although the present application has been described in detail with reference to the foregoing examples, it will be understood by one of ordinary skill in the art that the technical solutions described in the foregoing examples can still be modified or some technical features can be equivalently substituted; however, these modifications or substitutions do not make the essence of the corresponding technical solutions departing from the spirit and scope of the technical solutions of various examples of the present application.

### INDUSTRIAL APPLICABILITY

The present application discloses a method for preparing 3-chlorobicyclo[3.2.1]-3-octen-2-ol, wherein the method for preparing 3-chlorobicyclo[3.2.1]-3-octen-2-ol can be achieved by any of the following reaction routes: Reaction route I: 3,4-dichlorobicyclo[3.2.1]-2-octene is esterified in the presence of a carboxylate, and hydrolyzed in a strong base to obtain 3-chlorobicyclo[3.2.1]-3-octen-2-ol; Reaction route II: in the presence of an inorganic salt of strong base and weak acid, 3,4-dichlorobicyclo[3.2.1]-2-octene is reacted in a solvent to give 3-chlorobicyclo[3.2.1]-3-octen-2-ol. The present application can result in decreased formation of polymers and impurities, more obvious layering, and improved purification efficiency.

What is claimed is:

1. A method for preparing 3-chlorobicyclo[3.2.1]-3-octen-2-ol, wherein the method is obtained using any of following reaction routes:
reaction route I: 3,4-dichlorobicyclo[3.2.1]-2-octene is esterified in a presence of a carboxylate and hydrolyzed in a strong base to obtain 3-chlorobicyclo[3.2.1]-3-octen-2-ol;
reaction route II: in a presence an inorganic salt of strong base and weak acid, 3,4-dichlorobicyclo[3.2.1]-2-octene is reacted in a solvent to obtain 3-chlorobicyclo[3.2.1]-3-octen-2-ol.

2. The method according to claim 1, wherein in the esterification of the reaction route I, the carboxylate is selected from an alkali metal carboxylate or an alkali earth metal carboxylate, or the carboxylate is one or more selected from the group consisting of an alkali metal formate, an alkali metal acetate, an alkali metal propionate, an alkali metal 4-chlorobenzoate, an alkali metal benzoate, an alkali earth metal formate, an alkali earth metal acetate, an alkali earth metal propionate, an alkali earth metal 4-chlorobenzoate, and an alkali earth metal benzoate; or the carboxylate is one or more selected from the group consisting of sodium formate, sodium acetate, potassium acetate, calcium acetate, magnesium acetate, sodium propionate, potassium propionate, sodium 4-chlorobenzoate, potassium 4-chlorobenzoate, sodium benzoate, and potassium benzoate;
and/or, the molar ratio of 3,4-dichlorobicyclio[3.2.1]-2-octene to the carboxylate in the esterification of the reaction route I is 1:1-4, or 1:1.2-4, or 1:1.5-3.

3. The method according to claim 1, wherein a reaction temperature in the esterification reaction of the reaction route I is 0° C. to a reflux temperature of each solvent, or 50° C. to a reflux temperature of each solvent, or 70° C. to a reflux temperature of each solvent, or 80° C. to a reflux temperature of each solvent, or 90° C. to a reflux temperature of each solvent, or a reflux temperature of each solvent.

4. The method according to claim 2, wherein a reaction temperature in the esterification reaction of the reaction route I is 0° C. to a reflux temperature of each solvent, or 50° C. to a reflux temperature of each solvent, or 70° C. to a reflux temperature of each solvent, or 80° C. to a reflux temperature of each solvent, or 90° C. to a reflux temperature of each solvent, or a reflux temperature of each solvent.

5. The method according to claim 1, wherein a catalyst is further added in a mass ratio of 0-10% of a reaction system in each reaction, and the catalysts in each reaction are one or more independently or not independently selected from the group consisting of polyethylene glycol, tetrabutylammonium bromide, tetrabutylammonium chloride, benzyl trimethyl ammonium chloride, diisopropylethylamine, triethylamine, triethylenediamine, and crown ether.

6. The method according to claim 2, wherein a catalyst is further added in a mass ratio of 0-10% of a reaction system in each reaction, and the catalysts in each reaction are one or more independently or not independently selected from the group consisting of polyethylene glycol, tetrabutylammonium bromide, tetrabutylammonium chloride, benzyl trimethyl ammonium chloride, diisopropylethylamine, triethylamine, triethylenediamine, and crown ether.

7. The method according to claim 3, wherein a catalyst is further added in a mass ratio of 0-10% of a reaction system in each reaction, and the catalysts in each reaction are one or more independently or not independently selected from the group consisting of polyethylene glycol, tetrabutylammonium bromide, tetrabutylammonium chloride, benzyl trimethyl ammonium chloride, diisopropylethylamine, triethylamine, triethylenediamine, and crown ether.

8. The method according to claim 4, wherein a catalyst is further added in a mass ratio of 0-10% of a reaction system in each reaction, and the catalysts in each reaction are one or more independently or not independently selected from the group consisting of polyethylene glycol, tetrabutylammonium bromide, tetrabutylammonium chloride, benzyl trimethyl ammonium chloride, diisopropylethylamine, triethylamine, triethylenediamine, and crown ether.

9. The method according to claim 1, wherein the esterification reaction of the reaction route I is carried out in a solvent, and a mass ratio of 3,4-dichlorobicyclo[3.2.1]-2-octene to the solvent is 1:1-20, or 1:1-12.

10. The method according to claim 2, wherein the esterification reaction of the reaction route I is carried out in a solvent, and a mass ratio of 3,4-dichlorobicyclo[3.2.1]-2-octene to the solvent is 1:1-20, or 1:1-12.

11. The method according to claim 9, wherein the solvent in the esterification reaction is water or an organic solvent that is one or more selected from the group consisting of dimethylformamide, dimethylacetamide, ethylene glycol monomethyl ether, ethylene glycol dimethyl ether, ethylene glycol diethyl ether, N-methyl pyrrolidone, dimethyl sulfoxide, and sulfolane; when the esterification reaction is carried out in an organic solvent, all or part or none of the organic solvent is distilled out after the esterification reaction, and the hydrolysis reaction is carried out by adding water and a strong base after the esterification reaction;
and/or, in the reaction route I, a strong base is added for hydrolysis after complete esterification, alternatively, the carboxylate and the strong base are added to a reaction system at the same time for carrying out the esterification and hydrolysis reactions.

12. The method according to claim 11, wherein when the carboxylate and the strong base are added to the reaction system at the same time, a molar ratio of 3,4-dichlorobicyclo[3.2.1]-2-octene to the strong base is less than or equal to 1:1.

13. The method according to claim 10, wherein the solvent in the esterification reaction is water or an organic solvent that is one or more selected from the group consisting of dimethylformamide, dimethylacetamide, ethylene glycol monomethyl ether, ethylene glycol dimethyl ether, ethylene glycol diethyl ether, N-methyl pyrrolidone, dimethyl sulfoxide, and sulfolane; when the esterification reaction is carried out in an organic solvent, all or part or none of the organic solvent is distilled out after the esterification reaction, and the hydrolysis reaction is carried out by adding water and a strong base after the esterification reaction;
and/or, in the reaction route I, a strong base is added for hydrolysis after complete esterification, alternatively, the carboxylate and the strong base are added to a reaction system at the same time for carrying out the esterification and hydrolysis reactions.

14. The method according to claim 13, wherein when the carboxylate and the strong base are added to the reaction system at the same time, a molar ratio of 3,4-dichlorobicyclo[3.2.1]-2-octene to the strong base is less than or equal to 1:1.

15. The method according to claim 1, wherein the strong base in the hydrolysis reaction of the reaction route I is an alkali metal hydroxide and/or an alkali earth metal hydroxide; or, the strong base in the hydrolysis reaction of the reaction route I is one or more of sodium hydroxide, potassium hydroxide, lithium hydroxide, calcium hydroxide, and magnesium hydroxide;
and/or, a molar ratio of 3,4-dichlorobicyclo[3.2.1]-2-octene to the strong base in the hydrolysis reaction of the reaction route I is 1:0.5-2, or 1:1-2, or 1:1-1.5.

16. The method according to claim 2, wherein the strong base in the hydrolysis reaction of the reaction route I is an alkali metal hydroxide and/or an alkali earth metal hydroxide; or, the strong base in the hydrolysis reaction of the reaction route I is one or more of sodium hydroxide, potassium hydroxide, lithium hydroxide, calcium hydroxide, and magnesium hydroxide;
and/or, a molar ratio of 3,4-dichlorobicyclo[3.2.1]-2-octene to the strong base in the hydrolysis reaction of the reaction route I is 1:0.5-2, or 1:1-2, or 1:1-1.5.

17. The method according to claim 1, wherein the inorganic salt of strong base and weak acid in the reaction route II is one or more selected from the group consisting of carbonate, phosphate, and hydrogen phosphate; alternatively, the inorganic salt of strong base and weak acid is one or more of alkali metal carbonate, alkali metal phosphate, and alkali metal hydrogen phosphate; or alternatively, the inorganic salt of strong base and weak acid is one or more of sodium carbonate, potassium carbonate, tripotassium phosphate, and dipotassium hydrogen phosphate;
and/or, a molar ratio of 3,4-dichlorobicyclo[3.2.1]-2-octene to the inorganic salt of strong base and weak acid in the hydrolysis reaction of the reaction route II is 1:1-4, or 1:1-3, or 1:1.5-3.

18. The method according to claim 1, wherein water is used as a solvent in the reaction route II, and a mass ratio of 3,4-dichlorobicyclo[3.2.1]-2-octene to water is 1:1-20, or 1:1-15, or 1:3-15.

19. The method according to claim 17, wherein water is used as a solvent in the reaction route II, and a mass ratio of 3,4-dichlorobicyclo[3.2.1]-2-octene to water is 1:1-20, or 1:1-15, or 1:3-15.

20. The method according to claim 1, wherein a reaction temperature in the reaction route II is 0° C. to a reflux temperature of the solvent, or 50° C. to a reflux temperature of the solvent, or 70° C. to a reflux temperature of the solvent, or 90° C. to a reflux temperature of the solvent, or a reflux temperature of the solvent.

21. The method according to claim 17, wherein a reaction temperature in the reaction route II is 0° C. to a reflux temperature of the solvent, or 50° C. to a reflux temperature of the solvent, or 70° C. to a reflux temperature of the solvent, or 90° C. to a reflux temperature of the solvent, or a reflux temperature of the solvent.
